(19) United States
(12) Patent Application Publication
(10) Pub. No.: US 2003/0118632 A1
(43) Pub. Date: Jun. 26, 2003

(54) Methods and compositions for treating carpal tunnel syndrome

(76) Inventors: Larry Caldwell, San Jose, CA (US); Bradley S. Galer, West Chester, PA (US)

Correspondence Address: BOZICEVIC, FIELD & FRANCIS LLP, 200 MIDDLEFIELD RD, SUITE 200, MENLO PARK, CA, 94025, US

(21) Appl. No.: 10/029,408

(22) Filed: Dec. 26, 2001

(51) Int. Cl. A61K 31/415; A61K 9/70; A61K 31/192

(52) U.S. Cl. 424/449; 514/406; 514/570

(57) ABSTRACT

Methods are provided for treating a host suffering from a condition resulting from pressure applied to the median nerve of the carpal tunnel, e.g. carpal tunnel syndrome. In the subject methods, an effective amount of a topical NSAID formulation is applied to the palmar dermal surface proximal to the target median nerve. Practice of the above methods results in an amelioration of at least one symptom associated with the condition being treated. Also provided are kits for practicing the subject methods.

### INTRODUCTION

1. Technical Field

The technical field of the invention is carpal tunnel syndrome.

2. Background of the Invention

Carpal tunnel syndrome is caused by pressure exerted on the median nerve at the wrist. The median nerve supplies sensation to the thumb-side of the palm, and to the thumb, index finger, middle finger, and the thumb-side of the ring finger. It also supplies movement to part of the hand. The nerve enters the hand through a gap formed by the wrist bones (called the carpal bones) and the tough membrane that holds the bones together (the transverse carpal ligament). This gap is called the carpal tunnel. The passageway is rigid, so swelling of any of the tissues in this area can cause compression of the nerve (a condition also known as entrapment of the nerve).

Carpal Tunnel Syndrome is found most often in women 30 to 60 years old, but it is also found in men and in all age groups. Some of the conditions associated with carpal tunnel syndrome include pregnancy, premenstrual syndrome (PMS), and menopause; this is probably because of hormone changes that cause fluid retention and swelling of the tissues. Other conditions associated with carpal tunnel syndrome include rheumatoid arthritis, renal failure, diabetes mellitus, acromegaly, hypothyroidism, multiple myeloma, obesity, recent tuberculosis, fungal infection, and high blood pressure. Injury or trauma to the area, including (but not limited to) repetitive movement of the wrists, can cause swelling of the tissues and carpal tunnel syndrome. This injury may be from sports such as racquetball and handball, or from sewing, typing, driving, assembly-line work, painting, writing, use of tools (especially hand tools or tools that vibrate), repetitive stress or movement, or similar activities.

Carpal tunnel syndrome is characterized by the presence of one or more of the following symptoms: (a) weakness in 1 or both hands; (b) numbness or tingling or pain in the thumb and next 2 or 3 fingers of 1 or both hands; (c) numbness or tingling or pain of the palm of the hand joint pain (wrist pain) in 1 or both hands; (d) impaired fine finger movements in 1 or both hands; (e) weak grip; and (f) difficulty bringing the thumb across the palm to meet the other fingers (thumb opposition).

Treatment for carpal tunnel syndrome varies depending on the severity of the condition. Treatment options included resting and lifestyle changes; bracing and immobilization; oral diuretics and/or NSAIDS; system corticosteroids; injection of the carpal tunnel with corticosteroids; and surgery.

Because of the increasing prevalence of carpal tunnel syndrome, particularly due to increasing work related repetitive stress injury, there is a continued interest in the identification of new methods for treating this condition. Of interest would be the development of a simple method for at least ameliorating the pain associated with carpal tunnel syndrome that could be self-administered and would not have systemic effects.

3. Relevant Literature

Patents of interest include: U.S. Pat. Nos. 4,710,497; 4,740,374; 4,777,046; 4,956,171; 5,204,119; 5,373,022; 5,429,590; 5,695,779; and EPB 0574255. See also: Devi & Paranjothy, Drug Dev Ind Pharm (May 1999) 25: 695-700; Hui, et al., Pharm. Res., (October 1998)15:1589-95; Muller et al., J. Rheumatol. (September 1998) 25:1833-6; Burnham, et al., Clin J Sport Med (April 1998) 8: 78-81; Cordero, et al., J Pharm Sci (April 1997) 86; 503-8.

### SUMMARY OF THE INVENTION

Methods are provided for treating a host suffering from symptoms associated with pressure applied to the median nerve present in the carpal tunnel. In the subject methods, a topical NSAID formulation is applied to the palmar dermis proximal to the target median nerve for a period of time sufficient for at least an amelioration in one of the symptoms caused by the target median nerve. The topical NSAID formulation may be in any convenient topical formulation, such as a cream or patch, and includes a nonsalicylate NSAID in many embodiments of the invention. Also provided are compositions and kits for practicing the subject methods.

### DESCRIPTION OF THE SPECIFIC EMBODIMENTS

Methods for treating a host suffering from symptoms associated with pressure applied to the median nerve are provided. In the subject methods, a topical NSAID formulation is applied to the palmar dermis of the host proximal to the target median nerve. Topical application of the NSAID formulation results in at least an amelioration of at least one of the symptoms experienced by the host. Also provided are kits for practicing the subject methods.

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an” and “the” include plural reference unless the context clearly dictates otherwise. Unless defined otherwise, all technical and scientific terms used herein have the same meaning as commonly understood to one of ordinary skill in the art to which this invention belongs.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing the components which are described in the publications which might be used in connection with the presently described invention.

Methods

As summarized above, the subject invention provides a method for treating a host suffering from symptoms associated with pressure applied to the median nerve present in the carpal tunnel. The pressure on the median nerve that causes the pain treatable according to the subject methods may be the result of any of a number of causes. Representative causes include swelling of the tissues in the carpal tunnel, fluid retention, and the like.

As indicated above, topical NSAID formulations are employed in the subject methods. Any convenient topical formulation which provides for the requisite penetration of the active NSAID agent into the carpal tunnel area below the palmar dermis may be employed. The topical formulation may be a gel, cream, patch, or some other topical formulation. Topical formulations of NSAIDS are known in the art, where suitable formulations include those described in: U.S. Pat. Nos. 4,670,254; 4,710,497; 4,740,374; 4,777,046; 4,956,171; 4,999,379; 5,204,119; 5,373,022; 5,374,661; 5,429,590; 5,695,779; 5,814,599; and EPB 0574255, the disclosures of which are herein incorporated by reference. Of particular interest in many embodiments are cream or patch formulations.

The NSAIDs used in the present compositions are well known in the pharmaceutical art, are prepared via methods well known in the chemical and pharmaceutical arts, and include, for example, pharmaceutically active compounds having at least one acid moiety wherein such acid moiety is, most preferably, a carboxylic acid. Other acid moieties are well known to one of ordinary skill in the art. Representative non-steroidal, anti-inflammatory drugs (NSAIDs) include, but are not limited to: diclofenac, fenoprofen, flurbiprofen, ibuprofen, indomethacin, ketoprofen, ketorolac, naproxen, sulindac, etodolac, tometin, diflunisal, mefenamic acid, meclofenamic acid, and flufenamic acid. This list of NSAIDs is presented solely for the purpose of exemplification and is not intended to be limiting. Other NSAIDs which are used in the compositions of the present invention, are described, in addition to their dose regimens, in well known references such as, for example, the Physician's Desk Reference and the Merck Index. In many embodiments, the NSAID active agent present in the topical NSAID formulation is a nonsalycilate NSAID, where representative nonsalicylate NSAIDS include: propionic acids, e.g. fenoprofen, flurbiprofen, ibuprofen, ketoprofen, naproxen, etc.; acetic acids, e.g. diclofenac, etodolac, indomethacin, ketorolac, sulindac, tolmetin, etc.; fenamates, e.g. meclofenamate, mefenamic acid, etc.; oxicams, e.g. piroxicam, etc.; nabumetone; indomethacin; and the like.

The topical NSAID formulation employed in the subject methods is a formulation that includes an effective amount of an NSAID active agent, as described above. While the amount of any particular NSAID active agent may vary, in many embodiments, the topical formulation includes from about 0.1 to about 5.0%, usually from about 0.5 to about 3.0% and more usually from about 0.5 to about 2.0% w/w of an active NSAID agent.

In practicing the subject methods, the topical NSAID formulation is applied to the palmar dermis of the host in a manner sufficient to provide for penetration of an effective amount of NSAID active agent to the carpal tunnel without systemic activity. In other words, the topical NSAID formulation is applied to the keratinized skin surface of the host at a region sufficiently proximal to the carpal tunnel to provide for sufficient penetration of the NSAID through the keratinized skin surface and to the carpal tunnel. Typically, the topical formulation is applied to the base of the palm of the hand.

The amount of composition applied will usually be sufficient to cover a majority of the region of skin overlying the carpal tunnel to ensure that a sufficient amount of active agent penetrates to the carpal tunnel so that the host experiences amelioration of at least one symptom associated with the pressure applied on the median nerve. The exact amount of topical composition that is applied may be determined empirically. Generally, the amount of composition applied will be sufficient to cover at least about 25%, usually at least about 50%, more usually at least about 75% of the base of the palm of the hand. For solutions, dispersions, gels, lotions, creams and the like, the composition will be spread over the region and a covering optionally applied thereto. For patches, an appropriate sized patch will be placed over the region comprising the skin site. In many embodiments, the patch is specifically shaped to cover the target skin surface.

The formulation is maintained in place for a period of time sufficient for the desired amelioration in symptoms to occur. Generally, the formulation is maintained in place for at least about 30 min, usually at least about 3 hr and more usually for at least about 8 hr, where the formulation may be maintained in place for as long as 12 hr, 24 hr or longer.

Upon application of the topical composition, the NSAID active agent rapidly penetrates the surface of the skin to reach the carpal tunnel and reduce at least one symptom arising from pressure applied to the median nerve. As such, application and maintenance of the topical NSAID formulation as described above results in at least an amelioration of at least one symptom that is caused by the pressure applied on the carpal tunnel median nerve. By “at least an amelioration” is meant at least a reduction in the magnitude of the symptom, including a complete cessation or removal of the symptom. Symptoms ameliorated by the subject methods include one or more of: (a) weakness in at least one hand; (b) numbness or tingling in the thumb and next 2 or 3 fingers of 1 or both hands; (c) numbness or tingling of the palm of the hand joint pain (wrist pain) in 1 or both hands; (d) impaired fine finger movements in 1 or both hands; (e) weak grip; (f) difficulty bringing the thumb across the palm to meet the other fingers (thumb opposition); (g) pain in the wrist and hand; and the like.

A feature of the subject methods is that they result in no or substantially no toxic side effects which are observed in systemic, e.g., oral, NSAID delivery mechanisms. As such, the subject methods results in no or substantially no nausea, vomiting, etc., as observed with other systemically administered NSAIDs.

Treatment according to the subject methods results in at least amelioration from one or more symptoms of the underlying condition, as described above, for a period of time of at least about 6 hrs, usually at least about 12 hrs and more usually at least about 16 hrs, or longer, e.g., 24 hrs, 48 hrs or longer, e.g., one week, several weeks, months etc.

Utility

The subject methods find use in a variety of applications, and particularly in the treatment of disease conditions associated with the presence of pressure applied to the median nerve. Of particular interest is the use of the subject invention for the treatment of carpal tunnel syndrome, where the carpal tunnel syndrome condition may be the result of a number of causes, including: pregnancy, premenstrual syndrome (PMS), menopause, rheumatoid arthritis, renal failure, diabetes mellitus, acromegaly, hypothyroidism, multiple myeloma, obesity, recent tuberculosis, fungal infection, high blood pressure, injury or trauma to the area, including (but not limited to) repetitive movement of the wrists, etc.

A variety of hosts are treatable according to the subject methods. Generally such hosts are “mammals” or “mammalian,” where these terms are used broadly to describe organisms which are within the class mammalia. Of particular interest is the treatment of primates with the subject methods, (e.g., humans, chimpanzees, and monkeys), where the subject methods are particularly suited for use in the treatment of humans suffering from one or more symptoms associated with pressure applied to the median nerve of the carpal tunnel.

Topical Pharmaceutical Compositions

Also provided are topical pharmaceutical compositions comprising an effective amount of an NSAID active agent as described above, where the topical composition is present in a configuration that is tailored for its use in the treatment of carpal tunnel syndrome according to the subject methods. For example, topical patch formulations and analogous structures are provided that are shaped specifically with respect to the target epidermal location of their intended application, e.g., to cover the requisite surface area of the target location as described above. The amount of active NSAID agent present in the formulation may vary depending on the nature of the formulation and the specific NSAID active agent, but in many embodiments ranges from about 0.1 to about 5.0%, usually from about 0.5 to about 3.0% and more usually from about 0.5 to about 2.0% w/w. The topical patch may be used in conjunction with a device for securing the patch in place, e.g., a wrist band or other analogous device.

Kits

Kits with NSAID topical formulations used in the subject methods, are provided. Conveniently, the topical formulations may be provided in a unit dosage format, which formats are known in the art. In such kits, in addition to the containers containing the formulation, e.g. unit doses, is an informational package insert describing the use of the subject formulations in the methods of the subject invention, i.e. instructions for using the subject unit doses to treat symptoms associated with pressure applied to the median nerve of the carpal tunnel. These instructions may be present on one or more of the packaging, a package insert, and the like. In addition, a securing means, e.g., such as a wrist band, for holding the patch in place relative to the topical application location, may be provided in the kit, where the NSAID patch may be an integral component of the wrist band.

The following examples are offered by way of illustration and not by way of limitation.

### Experimental

A 26 year old man with a work-related history of “carpal tunnel symptoms” was experiencing tingling, numbness and some pain in the fingers of both hands. He applied a patch containing 1.3% diclofenac epolamine to the plantar aspect of each wrist and wore the patches overnight. The following morning, all symptoms were absent, and remained absent for approximately three weeks when they recurred. Again, over-night treatment with the patches alleviated the symptoms for a period of weeks.

The patch employed in the above protocol is a marketed product under the tradename of “Neurodol Tissugel” in several European countries. It consists of a hydrogel adhesive spread on a polyester felt backing. The drug substance diclofenac epolamine, is a patented salf form of diclofenac which is suitable for penetration through the skin. The drug substance is dissolved in the matrix of hydrogen adhesive.

It is evident from the above results and discussion that improved methods of treating disease conditions associated with pressure applied to the median nerve of the carpal tunnel, e.g. carpal tunnel syndrome, are provided. The subject methods offer a convenient, non-surgical form of treatment which nonetheless provides for rapid amelioration of at least one symptom associated with the disease condition being treated. Furthermore, the subject are amenable to self administration and do not give rise to systemic side effects. As such, the subject invention provides for significant contribution to the art.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. A method for at least ameliorating a symptom associated with pressure applied to the median nerve of the carpal tunnel of a host, said method comprising:
topically applying an affective amount of a topical NSAID formulation to a palmar dermal surface of said subject proximal to said carpal tunnel;
whereby at least one symptom associated with pressure applied to the median nerve of said carpal tunnel is at least ameliorated.

2. The method according to claim 1, wherein said topical NSAID formulation comprises a nonsalicylate NSAID.

3. The method according to claim 1, wherein said topical formulation is a cream.

4. The method according to claim 1, wherein said topical formulation is a patch.

5. The method according to claim 1, wherein said host is a mammal.

6. A method of treating a mammal suffering from carpal tunnel syndrome, said method comprising:
topically applying a nonsalicylate NSAID formulation to a palmar dermis of said mammal for a period of time sufficient for amelioration of at least one symptom of said syndrome to occur;
whereby said mammal is treated.

7. The method according to claim 6, wherein said mammal is a human.

8. The method according to claim 6, wherein said formulation is a cream.

9. The method according to claim 6, wherein said formulation is a patch.

10. The method according to claim 6, wherein said symptom is pain.

11. A method for treating a human suffering from pain caused by pressure on the median nerve, said method comprising:
topically applying a nonsalicylate NSAID formulation to the palmar dermis proximal to said median nerve in a manner sufficient to at least reduce said pain;
whereby said human is treated.

12. The method according to claim 11, wherein said NSAID formulation is a cream.

13. The method according to claim 11, wherein said NSAID formulation is a patch.

14. The method according to claim 11, wherein said NSAID is an acetic acid.

15. The method according to claim 14, wherein said NSAID is diclofenac.

16. The method according to claim 11, wherein said NSAID is indomethacin.

17. The method according to claim 11, wherein said NSAID is ibuprofen.

18. The method according to claim 11, wherein said NSAID is ketoprofen.

19. A kit for use in treating a subject suffering from pain associated with carpal tunnel syndrome, said kit comprising:
a topical NSAID formulation; and
instructions for practicing the method according to claim 1.

20. The kit according to claim 19, wherein said NSAID formulation comprises a nonsalicylate NSAID.

21. The kit according to claim 19, wherein said formulation is a cream.

22. The kit according to claim 19, wherein said formulation is a patch.

23. The kit according to claim 19, wherein said NSAID is an acetic acid.
